(19) United States
(12) Patent Application Publication
(10) Pub. No.: US 2023/0363749 A1
(43) Pub. Date: Nov. 16, 2023

(54) BI-STABLE LEVER ASSEMBLY FOR A SURGICAL TOOL

(71) Applicant: Verb Surgical Inc., Santa Clara, CA (US)

(72) Inventor: Jose Luis de Cordoba MATILLA, Malaga (ES)

(21) Appl. No.: 17/741,869

(22) Filed: May 11, 2022

(51) Int. Cl. A61B 17/00

(52) U.S. Cl. CPC A61B 17/00234; A61B 2018/0063

(57) ABSTRACT

A surgical tool for a surgical robotic system, the surgical tool comprising: a surgical tool grasper operable to perform a surgical procedure; and a handle coupled to the surgical tool grasper and having a lever operable to actuate the surgical tool grasper to perform the surgical procedure, the lever configured to move about a first pivot point and coupled to a bi-stable latch assembly configured to move about a second pivot point, and wherein a position of the bi-stable latch assembly relative to a boundary line intersecting the first pivot point and the second pivot point causes the bi-stable latch assembly to latch the lever in a closed position or unlatch the lever.

### TECHNICAL FIELD

This disclosure relates generally to the field of robotic surgery and, more particularly, to energy devices, systems and methods having a bi-stable lever or latch assembly and an auto energy option.

### BACKGROUND

Minimally-invasive surgery (MIS), such as laparoscopic surgery, involves techniques intended to reduce tissue damage during a surgical procedure. For example, laparoscopic procedures typically involve creating a number of small incisions in the patient (e.g., in the abdomen), and introducing one or more tools, for example a surgical stapler and/or an energy device, and at least one endoscopic camera through the incisions into the patient. The surgical procedures are then performed by using the introduced tools, with the visualization aid provided by the camera. Generally, MIS provides multiple benefits, such as reduced patient scarring, less patient pain, shorter patient recovery periods, and lower medical treatment costs associated with patient recovery. In some embodiments, MIS may be performed with robotic systems that include one or more robotic arms for manipulating surgical instruments based on commands from an operator.

### SUMMARY

Aspects of the disclosure include surgical tools, for example energy tools, harmonic tools, staplers, or any other surgical tool or device having a handle with a lever latching mechanism to facilitate control and/or manipulation of the surgical tool (e.g., application of energy using an energy tool) or device by the surgeon. An “energy tool” or “energy device” as used herein is intended to refer to any surgical instrument that can be used to manipulate a tissue by applying energy during a surgical procedure. For example, an energy tool or device may be any surgical instrument that can emit an energy sufficient to cut, dissect, burn, seal, coagulate, desiccate, fulgurate and/or achieve homeostasis of the tissue upon contact with the tissue. The energy tool or device may apply energy in the form of high frequencies, radio frequencies, ultrasonic waves, microwaves, or the like. In some aspects, the energy tool may include a surgical tool grasper that is inserted into the patient to perform the surgical procedure and is connected to a handle having a lever or trigger that controls the grasper. For example, during operation, the surgeon may hold the handle and squeeze the lever or trigger to a closed position which causes the grasper to emit energy. The opposite operation, for example pushing of the lever or trigger away from the handle to an open position may terminate the application of energy. In some aspects, the lever or trigger may latch (or otherwise be secured) in the closed position during energy application and remain latched until the surgeon applies an opposite force pushing the lever or trigger away from the handle. The lever or trigger may then remain in the open position until the surgeon reapplies the squeezing force pulling the lever or trigger toward the handle back to the closed position. In this aspect, the lever or latch may be considered a bi-stable latch or lever, or otherwise be considered associated with a bi-stable latch assembly, in that it is considered stable (e.g., resting) in either of two states, namely the closed position or the open position. In other aspects, the lever or trigger may be capable of moving between a number of positions (e.g., between the open position and the closed position) without latching during energy application. Representatively, the lever, trigger or associated latch assembly may be prevented from latching, however, capable of actuating an energy switch such that energy application can occur at a number of different lever or trigger positions without latching. The handle and lever or trigger configuration including, for example latching, non-latching and/or optional energy application options or modes, may provide a number of advantages to the user including, but not limited to, less force required on trigger for energy application, more comfortable use, the option of latching or non-latching based on user desire, possibility of energy application without latching, or the like.

Representatively, in one aspect the disclosure is directed to a surgical tool for a surgical robotic system, the surgical tool comprising a surgical tool grasper operable to perform a surgical procedure; and a handle coupled to the surgical tool grasper and having a lever operable to actuate the surgical tool grasper to perform the surgical procedure, the lever configured to move about a first pivot point and coupled to a bi-stable latch assembly configured to move about a second pivot point, and wherein a position of the bi-stable latch assembly relative to a boundary line intersecting the first pivot point and the second pivot point causes the bi-stable latch assembly to latch the lever in a closed position or unlatch the lever. In one aspect, moving the lever about the first pivot point in a clockwise direction moves the bi-stable latch assembly to a latched position that secures the lever in the closed position. In some aspects, the closed position actuates the surgical tool grasper to perform the surgical procedure. In still further aspects, moving the lever about the first pivot point in a counterclockwise direction moves the bi-stable latch assembly to a non-latched position that unlatches the lever. In some aspects, the lever is in an open position when unlatched that terminates the surgical procedure. In some aspects, the bi-stable latch assembly may include a first segment coupled to a second segment at a j oint, and wherein the movement of the lever causes the first segment to move about the joint relative to the second segment and moves the joint to a position over or under the boundary line. A positon of the joint above the boundary line may cause the bi-stable latch assembly to latch the lever in the closed position. In some aspects, a position of the joint below the boundary line causes the bi-stable latch assembly to unlatch the lever. In some aspects, the lever is latched in the closed position only by the bi-stable latch assembly. In still further aspects, the surgical tool may be an energy tool and the surgical procedure may include an energy operation.

In another aspect, the disclosure is directed to an energy tool for a surgical robotic system, the energy tool comprising: a tool grasper operable to perform an energy operation; and a handle coupled to the tool grasper and comprising a lever operable to actuate the tool grasper to perform the energy operation in a first mode in which the lever is latched in a closed position and a second mode in which the lever is unlatched. In some aspects, the lever pivots about a first pivot point and is coupled to a latch assembly that pivots about a second pivot point, and wherein a position of the latch assembly relative to a boundary line intersecting the first pivot point and the second pivot point latches the lever in the first mode or unlatches the lever in the second mode. In some aspects, the positioning of the latch assembly over the boundary line latches the lever in the closed position and actuates the tool grasper to perform the energy operation. In still further aspects, positioning of the latch assembly under the boundary line unlatches the lever allowing the lever to transition between the closed position and an open position. The energy tool may also include a lever adjustor coupled to the handle that prevents the latch assembly from latching in the second mode. The lever adjustor may be a bar that is operable to translate between a first position that allows the latch assembly to latch the lever in the first mode and a second position that prevents the latch assembly from latching the lever in the second mode. In some aspects, an energy application switch may be coupled to the handle, and when actuated, the switch may cause the tool grasper to perform the energy operation. In some aspects, the lever adjustor bar in the second position is aligned with the energy application switch and a movement of the lever about the pivot point to the closed position causes the lever adjustor bar to contact the energy application switch and actuate the tool grasper to perform the energy operation. In some aspects, the energy tool may further include a third mode in which the energy application switch is directly controlled by the user to cause the tool grasper to perform the energy operation when the lever is unlatched. In some aspects, the tool may include a selection lever coupled to the housing to allow a user to select between the first mode and the second mode.

The above summary does not include an exhaustive list of all aspects of the present invention. It is contemplated that the invention includes all systems and methods that can be practiced from all suitable combinations of the various aspects summarized above, as well as those disclosed in the Detailed Description below and particularly pointed out in the claims filed with the application. Such combinations have particular advantages not specifically recited in the above summary.

### BRIEF DESCRIPTION OF THE DRAWINGS

FIG. 1 is an overview schematic of an operating room arrangement with a surgical robotic system.

FIG. 2 is a side perspective view of one aspect of surgical tool of a surgical robotic system.

FIG. 3 is a side perspective cut out view of another aspect of a surgical tool of a surgical robotic system having a lever in an open position.

FIG. 4 is a side perspective view of another aspect of a surgical tool of a surgical robotic system having a lever in a closed position.

FIG. 5 is a magnified cross-sectional schematic view of another aspect of a lever of a surgical tool of a surgical robotic system.

FIG. 6 is a magnified cross-sectional schematic view of another aspect of a lever of a surgical tool of a surgical robotic system.

FIG. 7 is a magnified cross-sectional schematic view of another aspect of a lever of a surgical tool of a surgical robotic system.

FIG. 8 is a magnified cross-sectional schematic view of another aspect of a lever of a surgical tool of a surgical robotic system.

FIG. 9 is a magnified cross-sectional schematic view of another aspect of a lever of a surgical tool of a surgical robotic system.

FIG. 10 is a side perspective view of another aspect of an energy tool of a surgical robotic system.

FIG. 11 is a block diagram of a computer portion of a surgical robotic system including an energy tool, in accordance with an aspect of the disclosure.

### DETAILED DESCRIPTION

In various embodiments, description is made with reference to the figures. However, certain embodiments may be practiced without one or more of these specific details, or in combination with other known methods and configurations. In the following description, numerous specific details are set forth, such as specific configurations, dimensions, and processes, in order to provide a thorough understanding of the embodiments. In other instances, well-known processes and manufacturing techniques have not been described in particular detail in order to not unnecessarily obscure the description. Reference throughout this specification to “one embodiment,” “an embodiment,” or the like, means that a particular feature, structure, configuration, or characteristic described is included in at least one embodiment. Thus, the appearance of the phrase “one embodiment,” “an embodiment,” or the like, in various places throughout this specification are not necessarily referring to the same embodiment. Furthermore, the particular features, structures, configurations, or characteristics may be combined in any suitable manner in one or more embodiments.

In addition, the terminology used herein is for the purpose of describing particular aspects only and is not intended to be limiting of the invention. Spatially relative terms, such as “beneath”, “below”, “lower”, “above”, “upper”, and the like may be used herein for ease of description to describe one element’s or feature’s relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (e.g., rotated 90 degrees or at other orientations) and the spatially relative descriptors used herein interpreted accordingly.

As used herein, the singular forms “a”, “an”, and “the” are intended to include the plural forms as well, unless the context indicates otherwise. It will be further understood that the terms “comprises” and/or “comprising” specify the presence of stated features, steps, operations, elements, and/or components, but do not preclude the presence or addition of one or more other features, steps, operations, elements, components, and/or groups thereof.

The terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

Moreover, the use of relative terms throughout the description may denote a relative position or direction. For example, “distal” may indicate a first direction away from a reference point, e.g., away from a user. Similarly, “proximal” may indicate a location in a second direction opposite to the first direction, e.g., toward the user. Such terms are provided to establish relative frames of reference, however, and are not intended to limit the use or orientation of any particular surgical robotic component to a specific configuration described in the various embodiments below.

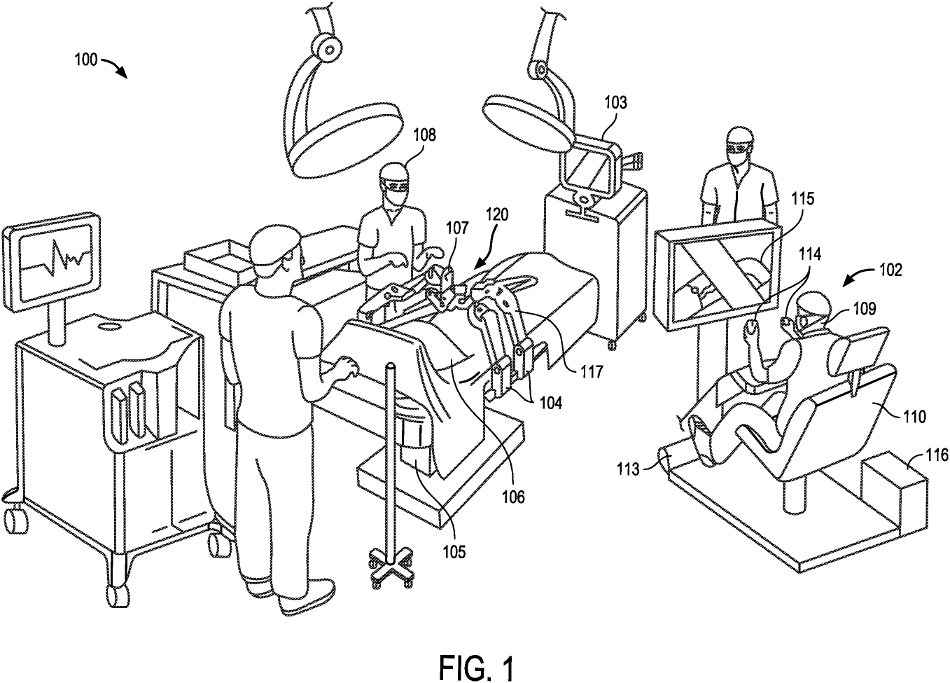

Referring to FIG. 1, this is a pictorial view of an example surgical robotic system 100 in an operating arena. The surgical robotic system 100 includes a user console 102, a control tower 103, and one or more surgical robots 120, including robotic arms 104 at a surgical robotic platform 105, e.g., an operating table, a bed, etc. The system 100 can incorporate any number of devices, tools, or accessories used to perform surgery on a patient 106. For example, the system 100 may include one or more surgical tools 107 used to perform surgery. A surgical tool 107 may be an end effector that is attached to a distal end of a surgical arm 104, for executing a surgical procedure. In some aspects, surgical tool 107 may include one or more of an energy tool, a harmonic tool, a stapler, or any other surgical tool or device.

Each surgical tool 107 may be manipulated manually, robotically, or both, during the surgery. For example, the surgical tool 107 may be a tool used to enter, view, or manipulate an internal anatomy of the patient 106. In an embodiment, the surgical tool 107 may be a grasper that can grasp tissue of the patient and/or an energy tool that can emit energy to cut, coagulate, desiccate and/or fulgurate the grasped tissue. The surgical tool 107 may be controlled manually, by a bedside operator 108; or it may be controlled robotically, via actuated movement of the surgical robotic arm 104 to which it is attached. The robotic arms 104 are shown as a table-mounted system, but in other configurations the arms 104 may be mounted in a cart, ceiling or sidewall, or in another suitable structural support.

Generally, a remote operator 109, such as a surgeon or other operator, may use the user console 102 to remotely manipulate the arms 104 and/or the attached surgical tools 107, e.g., teleoperation. Teleoperation may be engaged or disengaged based on the user actions. It should be understood that “engaging” the teleoperation mode is intended to refer to an operation in which, for example, a UID or foot pedal that is prevented from controlling the surgical instrument, is transitioned to a mode (e.g., a teleoperation mode) in which it can now control the surgical instrument. On the other hand, disengaging the teleoperation mode is intended to refer to an operation which occurs when the system is in a teleoperation mode, and then transitioned to a mode (non-teleoperation mode) in which the UID or foot pedal can no longer control the surgical instrument. For example, teleoperation mode may be disengaged when the system determines that a detected movement is an unintended action or movement by the user or the user engages in any other action which suggests teleoperation mode should no longer be engaged.

The user console 102 may be located in the same operating room as the rest of the system 100, as shown in FIG. 1. In other environments however, the user console 102 may be located in an adjacent or nearby room, or it may be at a remote location, e.g., in a different building, city, or country. The user console 102 may comprise a seat 110, one or more user interface devices, for example, foot-operated controls 113 or handheld user input devices (UID) 114, and at least one user display 115 that is configured to display, for example, a view of the surgical site inside the patient 106. In the example user console 102, the remote operator 109 is sitting in the seat 110 and viewing the user display 115 while manipulating a foot-operated control 113 and a handheld UID 114 in order to remotely control the arms 104 and the surgical tools 107 (that are mounted on the distal ends of the arms 104).

In some variations, the bedside operator 108 may also operate the system 100 in an “over the bed” mode, in which the bedside operator 108 (user) is now at a side of the patient 106 and is simultaneously manipulating a robotically-driven tool (end effector as attached to the arm 104), e.g., with a handheld UID 114 held in one hand, and a manual laparoscopic tool. For example, the bedside operator’s left hand may be manipulating the handheld UID to control a robotic component, while the bedside operator’s right hand may be manipulating a manual laparoscopic tool. Thus, in these variations, the bedside operator 108 may perform both robotic-assisted minimally invasive surgery and manual laparoscopic surgery on the patient 106.

During an example procedure (surgery), the patient 106 is prepped and draped in a sterile fashion to achieve anesthesia. Initial access to the surgical site may be performed manually while the arms of the robotic system 100 are in a stowed configuration or withdrawn configuration (to facilitate access to the surgical site). To create a port for enabling introduction of a surgical instrument into the patient 106, a trocar assembly may be at least partially inserted into the patient through an incision or entry point in the patient (e.g., in the abdominal wall). The trocar assembly may include a cannula or trocar, an obturator, and/or a seal. In some variations, the trocar assembly can include an obturator such as a needle with a sharpened tip for penetrating through a patient’s skin. The obturator may be disposed within the lumen of the cannula when being inserted into the patient 106, and then removed from the cannula such that a surgical instrument may be inserted through the lumen of the cannula. Once positioned within the body of the patient 106, the cannula may provide a channel for accessing a body cavity or other site within the patient 106, for example, such that one or more surgical instruments or tools (e.g., an energy tool) can be inserted into a body cavity of the patient 106, as described further herein. It will be understood that the cannula as described herein may be part of a trocar, and can optionally include an obturator or other components.

Once access is completed, initial positioning or preparation of the robotic system 100 including its arms 104 may be performed. Next, the surgery proceeds with the remote operator 109 at the user console 102 utilizing the foot-operated controls 113 and the UIDs 114 to manipulate the various end effectors and perhaps an imaging system, to perform the surgery. Manual assistance may also be provided at the procedure bed or table, by sterile-gowned bedside personnel, e.g., the bedside operator 108 who may perform tasks such as retracting tissues, performing manual repositioning, and tool exchange upon one or more of the robotic arms 104. Non-sterile personnel may also be present to assist the remote operator 109 at the user console 102. When the procedure or surgery is completed, the system 100 and the user console 102 may be configured or set in a state to facilitate post-operative procedures such as cleaning or sterilization and healthcare record entry or printout via the user console 102.

In one embodiment, the remote operator 109 holds and moves the UID 114 to provide an input command to move a robot arm actuator 117 in the robotic system 100. The UID 114 may be communicatively coupled to the rest of the robotic system 100, e.g., via a console computer system 116. Representatively, in some embodiments, UID 114 may be a portable handheld user input device or controller that is ungrounded with respect to another component of the surgical robotic system. For example, UID 114 may be ungrounded while either tethered or untethered from the user console. The term “ungrounded” is intended to refer to implementations where, for example, both UIDs are neither mechanically nor kinematically constrained with respect to the user console. For example, a user may hold a UID 114 in a hand and move freely to any possible position and orientation within space only limited by, for example, a tracking mechanism of the user console. The UID 114 can generate spatial state signals corresponding to movement of the UID 114, e.g. position and orientation of the handheld housing of the UID, and the spatial state signals may be input signals to control a motion of the robot arm actuator 117. The robotic system 100 may use control signals derived from the spatial state signals, to control proportional motion of the actuator 117. In one embodiment, a console processor of the console computer system 116 receives the spatial state signals and generates the corresponding control signals. Based on these control signals, which control how the actuator 117 is energized to move a segment or link of the arm 104, the movement of a corresponding surgical tool that is attached to the arm may mimic the movement of the UID 114. Similarly, interaction between the remote operator 109 and the UID 114 can generate for example a grip control signal that causes a jaw of a grasper of the surgical tool 107 to close and grip the tissue of patient 106.

The surgical robotic system 100 may include several UIDs 114, where respective control signals are generated for each UID that control the actuators and the surgical tool (end effector) of a respective arm 104. For example, the remote operator 109 may move a first UID 114 to control the motion of an actuator 117 that is in a left robotic arm, where the actuator responds by moving linkages, gears, etc., in that arm 104. Similarly, movement of a second UID 114 by the remote operator 109 controls the motion of another actuator 117, which in turn moves other linkages, gears, etc., of the robotic system 100. The robotic system 100 may include a right arm 104 that is secured to the bed or table to the right side of the patient, and a left arm 104 that is at the left side of the patient. An actuator 117 may include one or more motors that are controlled so that they drive the rotation of a joint of the arm 104, to for example change, relative to the patient, an orientation of an endoscope or a grasper of the surgical tool 107 that is attached to that arm. Motion of several actuators 117 in the same arm 104 can be controlled by the spatial state signals generated from a particular UID 114. The UIDs 114 can also control motion of respective surgical tool graspers. For example, each UID 114 can generate a respective grip signal to control motion of an actuator, e.g., a linear actuator, that opens or closes jaws of the grasper at a distal end of surgical tool 107 to grip tissue within patient 106. In some aspects, the surgical tool grasper may be a surgical stapler or energy tool and the UIDs 114 are used to control the opening or closing of the jaw of the surgical stapler or energy tool as well as the release of staples and/or energy application through the tissue. When the user is finished controlling the surgical tools with the UIDs 114, the user may dock (i.e., store) the UIDs 114 with docking stations or UID holders located on the console 102.

In some aspects, the communication between the platform 105 and the user console 102 may be through a control tower 103, which may translate user commands that are received from the user console 102 (and more particularly from the console computer system 116) into robotic control commands that are transmitted to the arms 104 on the robotic platform 105. The control tower 103 may also transmit status and feedback from the platform 105 back to the user console 102. The communication connections between the robotic platform 105, the user console 102, and the control tower 103 may be via wired and/or wireless links, using any suitable ones of a variety of data communication protocols. Any wired connections may be optionally built into the floor and/or walls or ceiling of the operating room. The robotic system 100 may provide video output to one or more displays, including displays within the operating room as well as remote displays that are accessible via the Internet or other networks. The video output or feed may also be encrypted to ensure privacy and all or portions of the video output may be saved to a server or electronic healthcare record system. It will be appreciated that the operating room scene in FIG. 1 is illustrative and may not accurately represent certain medical practices.

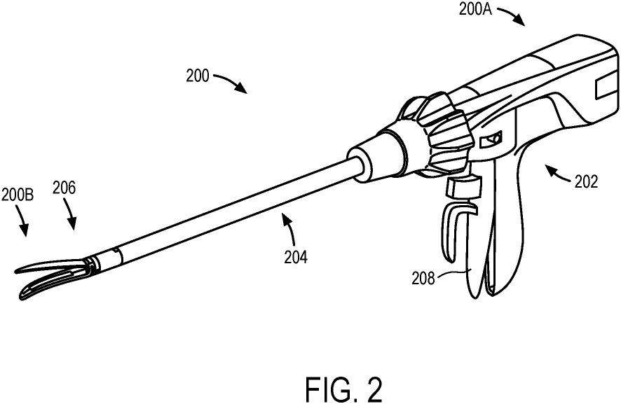

Turning now to FIG. 2, FIG. 2 illustrates a perspective view of one exemplary surgical tool or instrument, in this instance, an energy tool 200 for a surgical robotic system. Energy tool 200 may include a proximal end 200A that is held by the user outside of the patient during a surgical procedure and a distal end 200B that is inserted into the patient during a surgical procedure. Tool 200 may include a handle portion 202 a shaft portion 204 and a surgical tool grasper or jaw 206 coupled to the shaft portion 204. The handle portion 202 may include various mechanisms, for example a lever or trigger 208, suitable for manipulating the jaw 206 within the patient and controlling an energy application. For example, the lever or trigger 208 may be operable to transition between a closed position causing jaw 206 to perform a surgical operation (e.g., emission of energy from jaw 206) and an open position in which the surgical operation is terminated (e.g., no emission of energy from jaw 206). The shaft portion 204 may be an elongated portion that connects the handle portion 202 to the jaw 206. The shaft portion 204 may enclose circuitry or other components running from the handle portion 202 to jaw 206 for controlling the jaw 206 and the application of energy. The shaft portion 204 may be used to insert and position the jaw 206 within the patient.

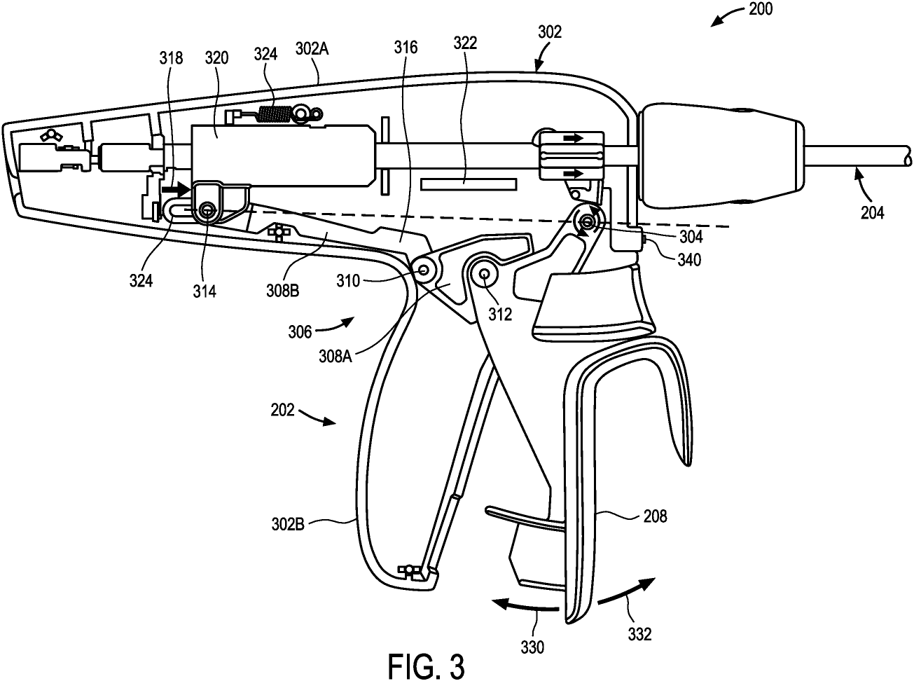

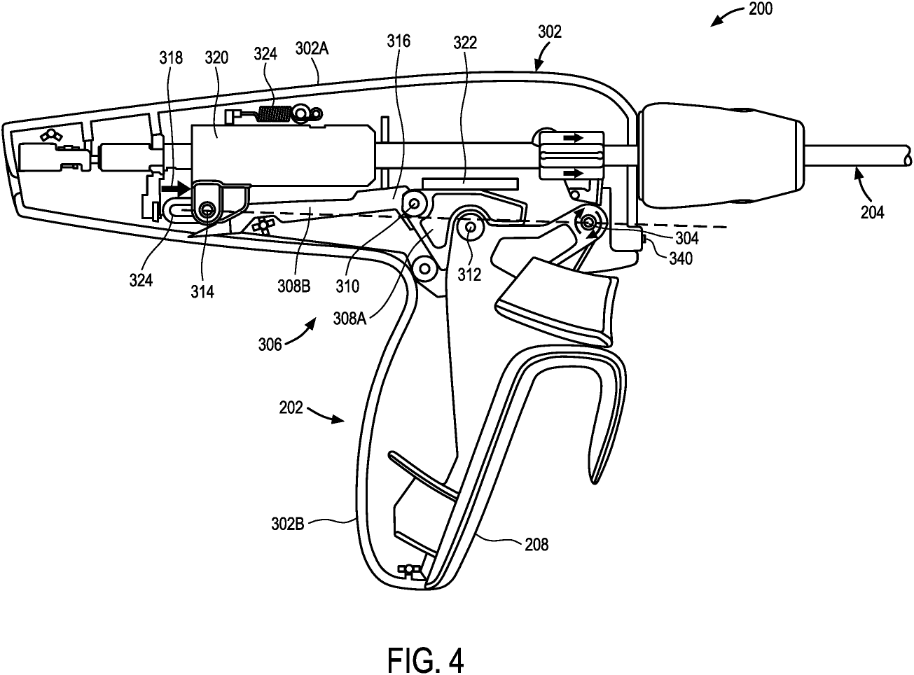

A number of representative surgical tool and lever configurations will now be discussed in more detail in reference to FIGS. 3-10. Representatively, FIG. 3 and FIG. 4 illustrate perspective views of a surgical tool handle having a bi-stable lever and latching assembly in a closed position (FIG. 3) or an open position (FIG. 4). Referring now to FIG. 3, FIG. 3 illustrates a side perspective view of handle 202 including lever 208 in an open position. It should be understood that although not shown in their entirety, handle 202 is coupled to the shaft portion 204 and jaw 206 as previously discussed to form the surgical tool 200. From this view, it can be seen that handle 202 includes a housing 302 that encloses or otherwise supports the various components (e.g., mechanical components, electrical components, or the like) used to operate the surgical tool. The housing 302 may form a body portion 302A and a base portion 302B of handle 202. Body portion 302A may be configured to house most of the electrical and mechanical tool components and be coupled to the shaft portion 204. Base portion 302B may extend from body portion 302A and be configured to rest within, near, or face, a palm of a user’s hand when the user is holding the handle 202.

Lever or trigger 208 is movably coupled to housing 302 in front of the base portion 302B such the user’s fingers wrap around, or otherwise contact, lever or trigger 208 when the user grasps handle 202. Lever or trigger 208 may be coupled to housing 302 at pivot point 304 which allows lever or trigger 208 to move relative to base portion 302A. Representatively, lever or trigger 208 may move, pivot or rotate about, pivot point 304 (e.g., a pivot joint or pin), for example, in a clockwise or counterclockwise direction. For example, lever 208 may move in a first direction as illustrated by arrow 330 to a first or closed position in which lever 208 contacts, or is otherwise near, base portion 302B. In some aspects, the first direction may be considered a clockwise direction around pivot point 304, a direction toward base portion 302B or any other direction which moves lever 208 toward base portion 302B to a position considered a closed position. Lever or trigger 208 may also move, pivot or rotate about, pivot point 304 (e.g., a pivot joint or pin), for example, in second direction as illustrated by arrow 332. For example, lever 208 may move in the second direction to a second or open position in which lever 208 is spaced a distance from base portion 302B. In some aspects, the second direction may be considered a counterclockwise direction, a direction away from base portion 302B or any other direction which moves lever 208 away from base portion 302B to a position considered an open position.

Lever or trigger 208 is further coupled to a latching assembly 306 that latches or unlatches the lever or trigger 208 in the closed and/or open positions. Representatively, in one position, configuration or mode, latching assembly 306 may latch (e.g., secure or hold) lever or trigger 208 in a closed position which enables energy activation and energy to be emitted from the tool. In some aspects, this position, configuration or mode in which latching assembly 306 latches lever or trigger 208 may be considered a latching or latched position. In another position, configuration or mode, latching assembly 306 may unlatch (e.g., release) lever or trigger 208 so it can transition to an open position. In the open position, energy activation may be terminated or disabled. In some aspects, this position, configuration or mode in which latching assembly 306 unlatches lever or trigger 208 may be considered a non-latching or non-latched position. In some aspects, latching assembly 306 and/or lever or trigger 208 may be considered stable in the closed position/configuration and the open position/configuration and therefore be referred to herein as bi-stable.

Referring now in more detail to latching assembly 306, latching assembly 306 may be an articulated joint including a first segment 308A that is movably connected to a second segment 308B by a pivot joint 310. In this aspect, first segment 308A can move (e.g., rotate or pivot) relative to second segment 308B about pivot joint 310. The other end of first segment 308A that is opposite the end coupled to pivot joint 310 may be fixedly coupled to lever 208. For example, the other end of first segment 308A may be coupled to lever 208 by a fastening member 312 (e.g., screw, bolt or the like). The other end of second segment 308B that is opposite the end coupled to the pivot joint 310 may be movably coupled to housing 302. For example, the other end of second segment 308B may be pivotally or rotatably coupled to housing 302 at pivot point or joint 314. In this aspect, a movement of lever 208 will cause the latching assembly 306 to also move. The latching assembly 306 may move to a position in which it either latches (e.g., secures) lever 208 at a particular position (e.g., closed position) or unlatches (e.g., releases) lever 208 so it may move to another position (e.g., open position).

Representatively, in some aspects, a movement of the latching assembly 308 to a configuration or position above/over or below/under a bi-stable boundary line 316 latches or unlatches the lever 308. The bi-stable boundary line 316 may be a line, axis or the like that is defined by, or otherwise intersects with, the pivot point 312 and pivot point 314. As can be seen from FIG. 3, when lever 208 is in the open position as illustrated (e.g. the user pushes lever 208 away from base portion 302B), first segment 308A and second segment 308B bend or pivot at pivot joint 310. This, in turn, causes pivot joint 310 to be positioned below or under bi-stable boundary line 316. Latching assembly 308 may remain in this position due to one or more springs 324 associated with pivot joint 314 that cause a biasing force in the direction of the arrow 318. For example, in some aspects, a spring 324 could be directly connected to pivot joint 314. In other aspects, where pivot joint 314 is attached to a yoke 320 of the tool (which drives operation of the grasper or jaw), a spring 324 which biases yoke 320 in the direction of arrow 318 may also indirectly bias pivot joint 314 in a direction of arrow 318. This biasing force may help hold latching assembly 308 in the illustrated position and prevent first and second segments 308A, 308B from bending at the pivot joint 310 in the absence of the application of an external force. In this position, latching assembly 308 is considered to be in a non-latched or unlatched position in which it does not latch lever 208 and lever 208 remains in the open (or unlatched) position. Latching assembly 308 and lever 208 are further considered stable in that they will remain in the illustrated position (e.g., non-latched or open position) until a force is applied to change their position.

Referring now to FIG. 4, when lever 208 moves to the closed position (e.g., the user squeezes lever 208 toward base portion 302B), first and second segments 308A, 308B of latching assembly 308 move, pivot or bend at the pivot joint 310. This, in turn, causes pivot joint 310 to move above, over or otherwise overpass bi-stable boundary line 316. The movement of pivot joint 310 above or over bi-stable boundary line 316 locks latching assembly 308 in the illustrated position (e.g., latched position) due to the previously discussed biasing force created by the spring 324. This, in turn, latches (or secures) lever 208 in the closed position as shown. Latching assembly 308 and lever 208 are further considered stable in that they will remain in the illustrated position (e.g., closed or latched position) until a force is applied to change their position. In some aspects, latching assembly 308 may be the only mechanism used to latch, hold or secure lever 208 in the closed position. For example, any other manual locking or latching mechanism on the handle 202 or lever 208 that the user must manually operate to latch lever 208 in a desired position is omitted.

The user may transition the lever 208 back to the open position by applying an opposite force, or otherwise pushing lever 208 away from base portion 302B. This force applied by the user overrides the biasing force holding latching assembly 308 in the latched position. This, in turn, causes latching assembly 308 to move below bi-stable boundary line 316 to the non-latched position in which it does not latch lever 208.

In addition, it should be understood that in some aspects, a stopper 322 may be coupled to housing 302, above latching assembly 308 and bi-stable boundary line 316. Stopper 322 may be of any size or shape, and at any location within housing 302, suitable for preventing latching assembly 308 from moving too far above bi-stable boundary line 316. Representatively, while it is desirable for pivot joint 310 of latching assembly 308 to move above or over bi-stable boundary line 316 to achieve the latched position, if pivot joint 310 moves too far above line 316 it may interfere with other tool operations or be difficult to transition back to the unlatched position. In this aspect, stopper 322 sets a maximum position above bi-stable boundary line 316 for pivot joint 310 that is suitable for latching the lever 208 as described without interfering with other tool operations and while still being operable to transition to the unlatched position (e.g., a position below bi-stable boundary line 316).

It should further be understood that the movement of lever 208 to the closed or open position causes the tool to emit energy, or otherwise perform a surgical operation or procedure, or terminate the energy application or surgical operation. In this aspect, lever 208 should be understood as being mechanically and/or electrically coupled to the surgical operation or procedure (e.g., energy application) of the tool using any conventional mechanism in which a movement of lever 208 to the closed position signals, or otherwise causes, the tool to perform a surgical operation (e.g., apply energy) and movement of lever 208 to the open position signals, or otherwise causes, the tool to terminate the surgical operation (e.g., energy application). For example, in some aspects, latching assembly 306 connects lever 208 to the tool components that operate the surgical tool grasper or jaw 206. Representatively, in some aspects the pivot joint 314 at the end of latching assembly 306 may be mechanically connected to a yoke assembly 320 which is, in turn, connected to circuitry or other components running from the handle portion 202 to jaw 206 for controlling the jaw 206 and the application of energy. In this aspects, the movement of latching assembly 306 in response to the movement of the lever 208 will also drive, or otherwise actuate, an operation of the jaw 206. In still further aspects, it is contemplated that other mechanisms, for example, a switch or button 340 placed near lever 208 may be used (e.g., pressed) to actuate or control the surgical procedure (e.g., energy application) in addition to, or instead of, lever 208. For example, the switch or button 340 could be associated with an energy function of the tool (e.g., mechanically or electrically associated) and when the user presses the switch or button 340, or otherwise moves the switch or button 340 to an “on” position, it causes the tool to emit energy, and when the switch or button 340 is not pressed or in an “off” position, it does not cause the tool to emit energy.

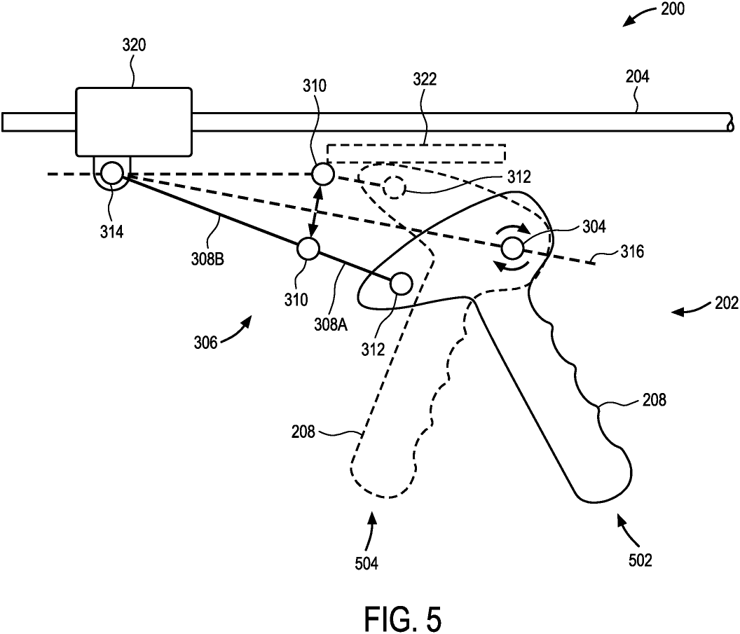

Referring now to FIG. 5, FIG. 5 illustrates a magnified cross-sectional side schematic view of a latching assembly and lever for a handle of a surgical tool transitioning between closed/latched and open/unlatched positions. It should be understood that some components of handle 202 are omitted, however, handle 202 should be understood as including the same components as previously discussed in reference to FIGS. 3-4. Representatively, lever 208 is shown transitioning between an open/unlatched position 502 and a closed/latched position 504 (illustrated in dashed lines). As can be seen from the illustration, when lever 208 is in the open/unlatched position 502, first and second segments 308A, 308B of latching assembly 306 rotate, bend or pivot relative to one another about pivot joint 310. The movement of first and second segments 308A, 308B causes pivot joint 310 to move to a position below bi-stable boundary line 316 as illustrated by the arrow. In this position below bi-stable boundary line 316, latching assembly 306 is considered to be in an unlatched position and lever 208 is further considered to be in an unlatched and stable open position. In other words, if the user were to release the force they are applying on the lever 208 (e.g., no longer push the lever) it would still remain in the open position. In addition, it can be understood that when lever 502 is in this open or unlatched position, it is not actuating or otherwise causing the surgical tool grasper to perform a surgical operation (e.g., application of energy).

On the other hand, when lever 208 pivots or rotates about pivot point 304 to the closed/latched position 504, first and second segments 308A, 308B of latching assembly 306 rotate, bend or pivot relative to one another about pivot joint 310 in an opposite direction. The movement of first and second segments 308A, 308B causes pivot joint 310 to move to a position above bi-stable boundary line 316 as illustrated by the arrow. In this position above bi-stable boundary line 316, latching assembly 306 is considered to be in a latched position and lever 208 is further considered latched and stable in the closed position. In other words, if the user were to release the force they are applying on the lever 208 (e.g., no longer squeeze the lever), latching assembly 306 and lever 208 would still remain in the latched or closed position. In addition, it can be understood that when lever 208 is in this closed or latched position, it is actuating or otherwise causing the surgical tool grasper to perform a surgical operation (e.g., application of energy). It can further be seen from this view that stopper 322 stops or otherwise prevents latching assembly 306 from moving farther then desired above bi-stable boundary line 316 and possibly interfering with or contacting the other handle component operations (e.g., operations associated with yoke 320 and/or shaft 204). In this aspect, stopper 322 may also be understood as a mechanism or component which sets or limits the range of movement of latching assembly 306 to a desired range.

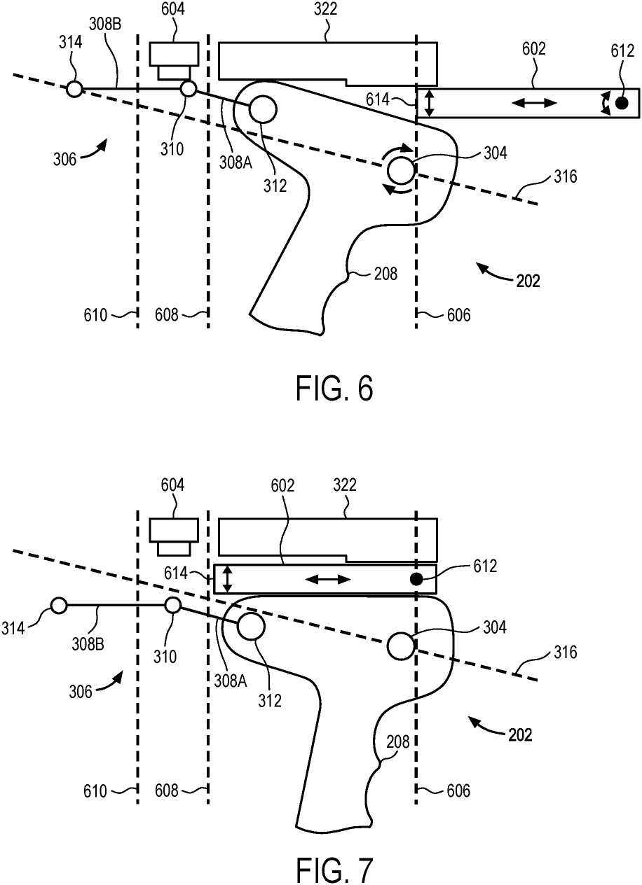

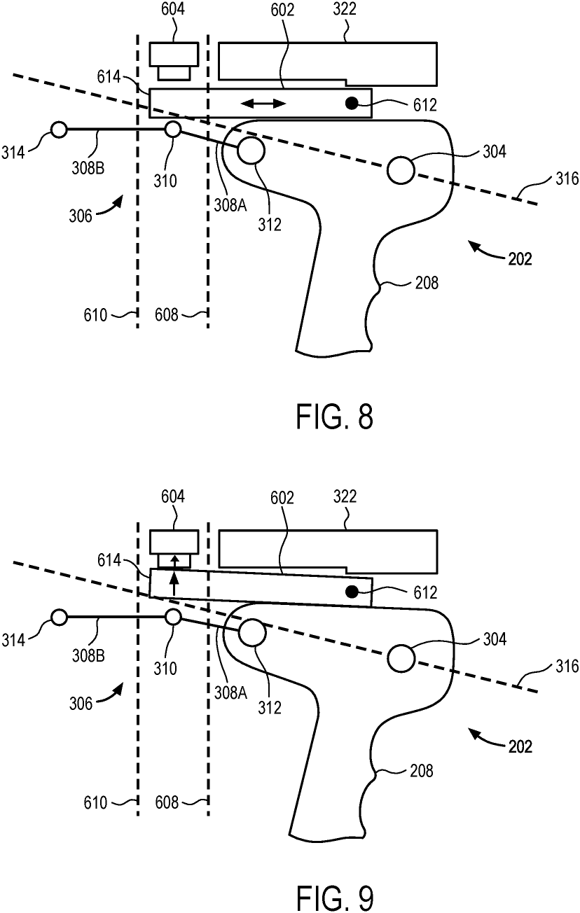

Referring now to FIG. 6, FIG. 7, FIG. 8, and FIG. 9, FIGS. 6-9 illustrate an alternative handle and lever configuration. Representatively, FIGS. 6-9 illustrate a handle 202 and lever 208 having an optional bi-stable mode and auto energy option. It should be understood that although some components of handle 202 are omitted, handle 202 illustrated in FIGS. 6-9 may include the same components as previously discussed in reference to FIGS. 3-4. Representatively, handle 202 may include lever 208 and latching assembly 306 including the same components as previously discussed. In addition, in this configuration, handle 202 may include a lever adjustor 602 and an energy application button or switch 602 to allow for an optional bi-stable mode and auto energy option. Representatively, lever adjustor 602 may be used to adjust an operation of lever 208 so that in one mode, lever 208 may be latched in a closed position while in another mode it is prevented from latching and can move to different positions.

In addition, lever adjustor 602 may connect lever 208 to energy application switch 604 so that even when lever 208 is prevented from latching, it can still be used for energy application. Representatively, lever adjustor 602 may be connected to the handle (e.g., housing 302) such that it can both translate as illustrated by the horizontal arrow and in some aspects also move up and down as illustrated by the vertical arrow. In some aspects, lever adjustor 602 may, for example, be a bar, rode or the like that is coupled to the handle housing at a position above the lever 208, for example, at a position between a top end of lever 208 and stopper 322. In some aspects, lever adjustor 602 may be coupled to a rail or other track like mechanism that guides or otherwise allows for the translation of lever adjustor 602 within the housing. Lever adjustor 602 may translate between different positions (or modes) that allow the latch assembly 306 to latch the lever 208, prevent the latch assembly 306 from latching the lever 208, and/or prevent the latch assembly 306 from latching the lever 208 but allow for energy application by contacting the energy application switch 604. The representative positions (or modes) of lever adjustor 602 are illustrated by dashed lines 606, 608 and 610. In still further aspects, an end 614 of lever adjustor 602 may move up and/or down by rotating about pivot point 612. In this aspect, when lever adjustor 602 is at a position (or mode) in which it is between the lever 208 and energy switch 604 (e.g., position 610), the movement of the lever 208 may move the end 614 of lever adjustor 602 upward causing it to contact and actuate the energy switch 604. Energy switch 604 may be any type of conventional switch, button or mechanism that is electrically and/or mechanically coupled to the energy function of the tool such that when it is pressed, or otherwise contacted, it can cause the tool to apply energy. When energy switch 604 is not pressed or otherwise in an “on” position, it will not activate the energy function, or otherwise cause energy to be emitted. In this aspect, even in an unlatched position, the user is able to control an energy operation using the lever 208.

Referring now in more detail to FIG. 6, FIG. 6 shows lever adjustor 602 in a position (or mode) 606. In some aspects, position (or mode) 606 may be considered a first position or mode. As can be seen from FIG. 6, when lever adjustor 602 is in position (or mode) 606, lever 208 is free to rotate about pivot point 304 to the open/latched position as previously discussed. In particular, at position (or mode) 606, the end 614 of lever adjustor 602 may be to the right of pivot point 304 (e.g., between pivot point 304 and shaft 204) such that it does not interfere with the range of movement of lever 208. For example, lever adjustor 602 may be at a position distal to pivot point 304 (e.g., between pivot point 304 and distal end 200B of the tool). Said another way, lever adjustor 602 is at a position where it does not prevent lever 208 from rotating or moving to the closed position (until it contacts stopper 322). It can further be seen that pivot joint 310 of latch assembly 306 is also able to move above bi-stable boundary line 316 so that it can latch or otherwise secure lever 208 in this closed (or latched) position as previously discussed. In addition, in the closed or latched position of lever 208 shown in FIG. 6, energy is applied by the tool as previously discussed.

FIG. 7 shows lever adjustor 602 in a position (or mode) 608. In some aspects, position (or mode) 608 may be considered a second position or mode. Representatively, from the position (or mode) 606 shown in FIG. 6, lever adjustor 602 translates (or slides) to the left so that end 614 is at a second position (or mode) 608. In position (or mode) 608, lever adjustor 602 is directly above lever 208 and directly below stopper 322. For example, lever adjustor 602 extends above lever 208 between pivot points 304, 312 of lever 208. The range of motion of lever 208 is therefore limited by the lever adjustor 602. In particular, lever 208 may only rotate about pivot point 304 until it contacts lever adjustor 602. Lever adjustor 602 therefore prevents lever 208 from rotating about pivot point 304 to the latched position shown in FIG. 6. In addition, lever 208 does not rotate far enough to cause the pivot joint 310 of latch assembly 306 to overpass bi-stable boundary line 316. Latch assembly 306 therefore does not latch or otherwise secure lever 208 in any particular position. Lever 208 is therefore free to move between the closed position shown in FIG. 7 and an open position as desired by the user. For example, the user may squeeze lever 208 toward the base of the handle as previously discussed to achieve the position shown in FIG. 7. In addition, as can be seen from FIG. 7, in some aspects, pivot joint 310 of latch assembly 306 may be at a position that is just below the bi-stable boundary line 316. Latch assembly 306 is therefore almost balanced or stable (e.g., at a semi-equilibrium position) in this position. This, in turn, causes lever 208 to also be almost balanced or stable and want to stay at this position. Lever 208 may therefore remain in this position without the user having to applying any force (or only minimal force). Such a configuration may be more comfortable to a user desiring a lever 208 with less resistance to movement and/or requiring less force applied by the user to control the tool operations.

In still further aspects, upon releasing the lever 208 (i.e. no longer squeezing), lever 208 may automatically move to a fully open position (e.g., position 502 shown in FIG. 5), for example, due to the spring forces biasing lever 208 toward the open position as previously discussed. In this aspect, the user has the option of using lever 208 in a mode in which it will latch (e.g., position or mode 606) or using lever 208 in a mode in which it will not latch (e.g., position or mode 608) depending on the user’s preference.

FIG. 8 shows lever adjustor 602 in another position (or mode) 610. In some aspects, position (or mode) 610 may be a third position or mode. Representatively, from the position (or mode) 608 shown in FIG. 7, lever adjustor 602 translates (or slides) to the left so the end 614 is at position (or mode) 610. For example, lever adjustor 602 may be at a position proximal to pivot point 304 (e.g., between pivot point 304 and proximal end 200A of the tool). In position (or mode) 610, lever adjustor 602 is directly above lever 208 and directly below stopper 322. The range of motion of lever 208 is therefore limited by the lever adjustor 602 as previously discussed. Representatively, lever adjustor 602 prevents lever 208 from rotating about pivot point 304 to a position which causes latch assembly 306 to overpass bi-stable boundary line 316. Latch assembly 306 therefore does not latch or otherwise secure lever 208 in any particular position. In addition, as previously discussed, in this position, lever 208 is in essentially a semi equilibrium position such that lever 208 will remain in the closed position without the user having to apply a force (or only minimal force). The lever 208 may therefore require less force to operate making it potentially more comfortable for the user.

In addition, in this position (or mode) 610, the end 614 of lever adjustor 602 extends beyond the end of stopper 322 and is positioned directly below energy application switch 604. For example, the end 614 of lever adjustor 602 may be positioned to the left of lever pivot point 312, for example, between pivot point 312 and pivot point 314 of latching assembly 306. As a result, when the user squeezes lever 208, lever 208 may contact lever adjustor 602 and cause the end 614 of lever adjustor 602 to move up and/or down. For example, when the user squeezes lever 208, the lever 208 pushes on lever adjustor 602 causing the end 614 to move in an upward direction as shown in FIG. 9. The movement of lever adjustor 602 upward, further causes lever adjustor 602 to press on (or otherwise contact) energy application switch 604. Pressing or contacting energy application switch 604 in this manner automatically activates the energy function of the tool such that, for example, energy is emitted from the surgical tool grasper.

Thus, in position (or mode) 610, lever 208 can be closed by the user and actuate an energy function of the tool, but without the lever 208 becoming latched in the closed position. Lever 208 is therefore free to move between the closed position shown in FIG. 9 and an open position as desired by the user. For example, the user may squeeze lever 208 toward the base of the handle as previously discussed to achieve the position shown in FIG. 9. Upon releasing the lever 208 (i.e. no longer squeezing), lever 208 may automatically move to a fully open position (e.g., position 502 shown in FIG. 5), for example, due to the spring forces biasing lever 208 toward the open position as previously discussed. In this aspect, the user has the option of using lever 208 in a mode in which it will not latch (e.g., position or mode 610) but can still be used to activate the energy function of the tool. This option or mode in which lever 208 does not need to latch while still being able to apply energy may be desirable to some users who prefer the feel or function of a lever 208 that does not latch, or may otherwise have less resistance to movement by the user.

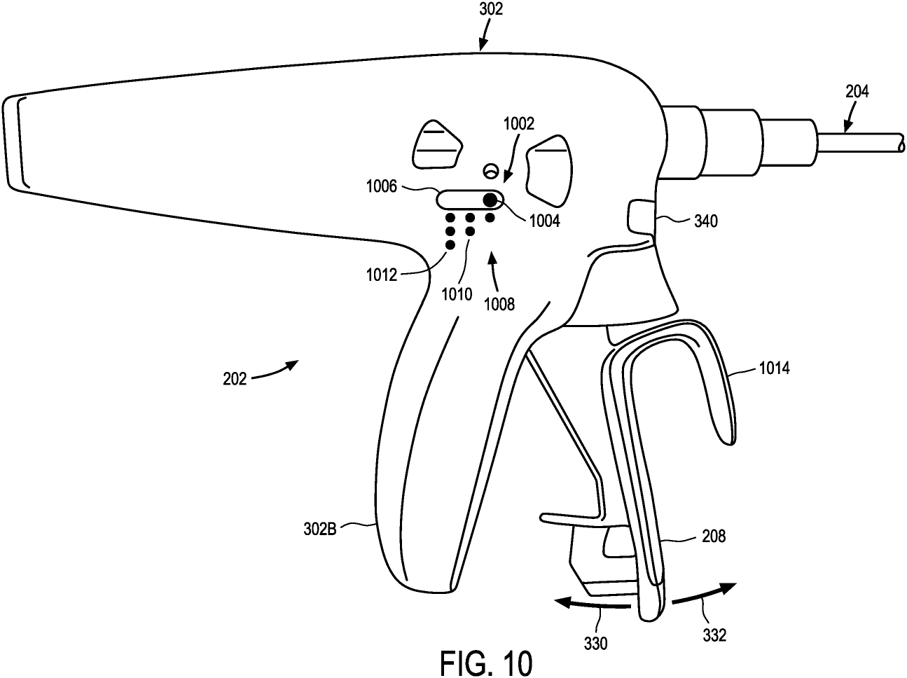

FIG. 10 is a side perspective view of a surgical tool handle as previously discussed, and including a selection lever to control or otherwise allow the user to select a mode of the lever. In particular, FIG. 10 illustrates a surgical tool 200 including a handle 202 coupled to a shaft 204 that connects to the surgical tool grasper or jaw (e.g., grasper or jaw 206) as previously discussed. Handle 202 is shown including housing 302 that forms a base portion 302B to which a lever or trigger 208 is attached to control a surgical operation (e.g., energy application) of the tool grasper or jaw. Lever 208 may be the same lever configuration as described in reference to FIGS. 6-9 and be operably connected to a latching assembly (e.g., latching assembly 306) and lever adjustor (e.g., lever adjustor 602) that allow for lever 208 to operate in different modes (e.g., latching mode, non-latching mode, and/or non-latching mode with energy activation).

FIG. 10 further illustrates a selection lever 1002 that allows the user to select between the different modes of operation. Representatively, selection lever 1002 may include a selection knob, switch, pin 1004, or other similar structure, that a user may manipulate to transition between the different modes. For example, in one aspect, pin 1004 may be configured to slide within a guide or slot 1006 of housing 302 to at least three different locations or positions 1008, 1010 and 1012. In some aspects, pin 1004 may be configured to slide 1 centimeter or 2 centimeters between the different locations or positions 1008, 1010 and 1012. The sliding movement can be done manually by the user, or could be done with an internal motor (e.g., a micro-motor). Each of the different locations or positions 1008, 1010 and 1012 may correspond to a different mode of operation. In some aspects, pin 1004 may be mechanically connected to the previously discussed lever adjustor 602 such that sliding of the pin 1004 also translates lever adjustor 602 to the different positions previously discussed in reference to FIGS. 6-9. In other aspects, however, it is contemplated that selection lever 1002 and/or pin 1004 may be indirectly connected, or electrically connected, to lever adjustor 602 such that, for example, the position of pin 1004 signals to the tool to operate in the desired mode.

Representatively, sliding pin 1004 to position 1008 will cause the tool to operate in a latched or latching mode. For example, position 1008 may correspond to the mode illustrated by FIG. 6 in which lever adjustor 602 is at position 606 and lever 208/latching assembly 306 pivot or rotate so that latching assembly 306 is above the bi-stable boundary line 316 and latches lever 208 in the closed position. In addition, as previously discussed, in this configuration, lever 208 is also stable in the open position. Therefore, this latched or latching mode may also be referred to as a bi-stable mode since in this mode, lever 208 is stable in both the closed and open positions as previously discussed. In addition, in this mode the latching of the lever 208 will cause energy activation and therefore energy to be emitted by the tool.

In another aspect, sliding pin 1004 to position 1010 will cause the tool to operate in a non-latching or non-latched mode, or mode in which latching is considered disabled. For example, position 1010 corresponds to the mode illustrated by FIG. 7 in which lever adjustor 602 is at position 608 and lever 208/latching assembly 306 have a limited range of motion and are prevented from overpassing, or otherwise moving above, the bi-stable boundary line 316. In this aspect, latching assembly 306 will not latch lever 208 in the closed position and lever 208 continues to be free to move between open and closed positions as desired by the user. In addition, in this mode, lever 208 may still be used to, for example manipulate the grasper or jaw to open and/or close, but may not cause energy activation. Rather, handle 200 may include a separate button, switch or other mechanism that the user can press or otherwise manipulate to activate the energy function. For example, an energy application switch or button 340 near lever 208 as shown in FIG. 10.

In still further aspects, sliding pin 1004 to position 1012 will cause the tool to operate in a non-latching mode, or mode in which latching is considered disabled, but also with an energy activation option. For example, position 1012 corresponds to the modes illustrated by FIGS. 8-9 in which lever adjustor 602 is at position 610 and lever 208/latching assembly 306 have a limited range of motion and are prevented from overpassing, or otherwise moving above, the bi-stable boundary line 316. In this aspect, latching assembly 306 will not latch lever 208 in the closed position and lever 208 continues to be free to move between open and closed positions as desired by the user. In addition, in this mode, the lever adjustor 602 is aligned with the energy application switch 604. Thus, the movement of lever 208 toward the handle will cause lever adjustor 602 to contact energy application switch 604. Contacting energy application switch 604 will, in turn, actuate energy application. Therefore, in this mode, even though lever 208 does not latch, it can still be used to activate the energy function and control the tool.

It should further be understood that while three locations or positions 1008, 1010, 1012 corresponding to three modes are described, the locations or positions 1008, 1010, 1012 may correspond to other modes and/or there may be more or less locations or positions used to select the different modes. In addition, other mechanisms suitable for mode selection may be used to transition the tool to the different modes disclosed herein and the tool is not intended to be limited to the particular selection lever and pin configuration disclosed herein.

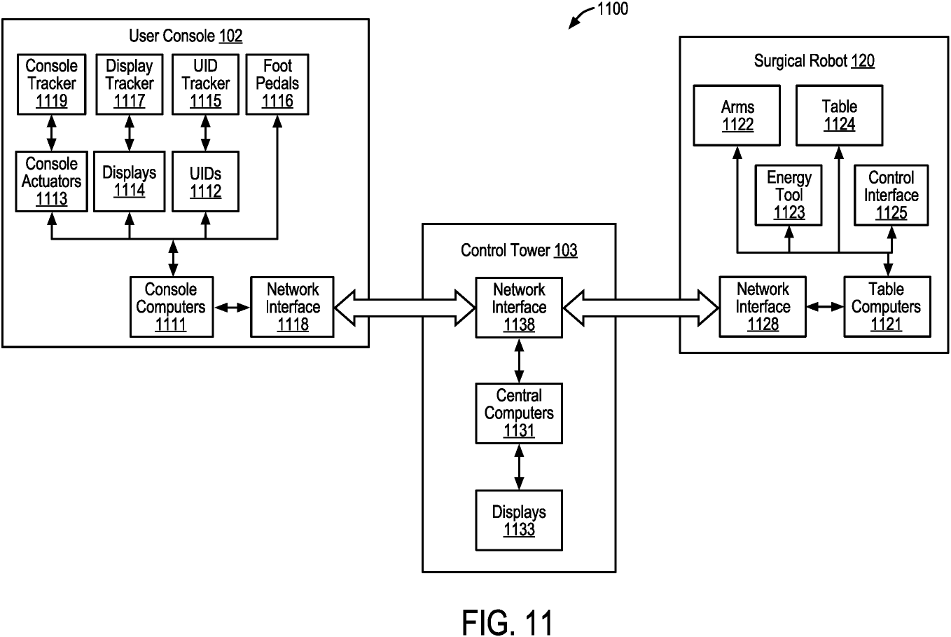

FIG. 11 is a block diagram of a computer portion of a surgical robotic system, which is operable to implement any one or more of the previously discussed operations. The exemplary surgical robotic system 1100 may include a user console 102, a surgical robot 120, and a control tower 103.The surgical robotic system 1100 may include other or additional hardware components; thus, the diagram is provided by way of example and not a limitation to the system architecture.

As described above, the user console 102 may include console computers 1111, one or more UIDs 1112, console actuators 1113, displays 1114, foot pedals 1116 and a network interface 1118. In addition, user console 102 may include a number of components, for example, a UID tracker(s) 1115, a display tracker(s) 1117 and a console tracker(s) 1119, for detecting various surgical conditions required for operation of the system (e.g., UID orientation, orientation of the surgeon relative to the display, orientation the console seat, etc.). It should further be understood that a user or surgeon sitting at the user console 102 can adjust ergonomic settings of the user console 102 manually, or the settings can be automatically adjusted according to user profile or preference. The manual and automatic adjustments may be achieved through driving the console actuators 1113 based on user input or stored configurations by the console computers 1111. The user may perform robot-assisted surgeries by controlling the surgical robot 120 using one or more master UIDs 1112 and foot pedals 1116. Positions and orientations of the UIDs 1112 are continuously tracked by the UID tracker 1115, and status changes are recorded by the console computers 1111 as user input and dispatched to the control tower 103 via the network interface 1118. Real-time surgical video of patient anatomy, instrumentation, and relevant software apps can be presented to the user on the high resolution 3D displays 1114 including open or immersive displays.

The user console 102 may be communicatively coupled to the control tower 103. The user console also provides additional features for improved ergonomics. For example, the user console may be an open architecture system including an open display, although an immersive display, in some cases, may be provided. Furthermore, a highly-adjustable seat for surgeons and master UIDs tracked through electromagnetic or optical trackers are included at the user console 102 for improved ergonomics.

The control tower 103 can be a mobile point-of-care cart housing touchscreen displays, computers that control the surgeon’s robotically-assisted manipulation of instruments, safety systems, graphical user interface (GUI), light source, and video and graphics computers. As shown in FIG. 11, the control tower 103 may include central computers 1131 including at least a visualization computer, a control computer, and an auxiliary computer, various displays 1133 including a team display and a nurse display, and a network interface 1138 coupling the control tower 103 to both the user console 102 and the surgical robot 120. The control tower 103 may offer additional features for user convenience, such as the nurse display touchscreen, soft power and E-hold buttons, user-facing USB for video and still images, and electronic caster control interface. The auxiliary computer may also run a real-time Linux, providing logging/monitoring and interacting with cloud-based web services.

The surgical robot 120 may include an operating table 1124 with a plurality of integrated robotic arms 1122 that can be positioned over the target patient anatomy. An energy tool 1123 can be attached to or detached from the distal ends of the arms 1122, enabling the surgeon to perform various surgical procedures. The energy tool 1123 may be any one or more of the energy tools having sensors integrated therein as previously discussed in reference to FIG. 2-FIG. 10. The surgical robot 120 may also comprise control interface 1125 for manual or automated control of the arms 1122, table 1124, and tools 1123. The control interface can include items such as, but not limited to, remote controls, buttons, panels, and touchscreens. Other accessories such as trocars (sleeves, seal cartridge, and obturators) and drapes may also be needed to perform procedures with the system. In some variations, the plurality of the arms 1122 includes four arms mounted on both sides of the operating table 1124, with two arms on each side. For certain surgical procedures, an arm mounted on one side of the table can be positioned on the other side of the table by stretching out and crossing over under the table and arms mounted on the other side, resulting in a total of three arms positioned on the same side of the table 1124. The surgical tool can also comprise table computers 1121 and a network interface 1128, which can place the surgical robot 120 in communication with the control tower 103.

The foregoing description, for purposes of explanation, used specific nomenclature to provide a thorough understanding of the invention. However, it will be apparent to one skilled in the art that specific details are not required in order to practice the invention. Thus, the foregoing descriptions of specific aspects of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, and they thereby enable others skilled in the art to best utilize the invention and various embodiments with various modifications as are suited to the particular use contemplated.

What is claimed is:

1. A surgical tool for a surgical robotic system, the surgical tool comprising:
a surgical tool grasper operable to perform a surgical procedure; and
a handle coupled to the surgical tool grasper and having a lever operable to actuate the surgical tool grasper to perform the surgical procedure, the lever configured to move about a first pivot point and coupled to a bi-stable latch assembly configured to move about a second pivot point, and wherein a position of the bi-stable latch assembly relative to a boundary line intersecting the first pivot point and the second pivot point causes the bi-stable latch assembly to latch the lever in a closed position or unlatch the lever.

2. The surgical tool of claim 1 wherein moving the lever about the first pivot point in a clockwise direction moves the bi-stable latch assembly to a latched position that secures the lever in the closed position.

3. The surgical tool of claim 1 wherein the closed position actuates the surgical tool grasper to perform the surgical procedure.

4. The surgical tool of claim 1 wherein moving the lever about the first pivot point in a counterclockwise direction moves the bi-stable latch assembly to a non-latched position that unlatches the lever.

5. The surgical tool of claim 1 wherein the lever is in an open position when unlatched that terminates the surgical procedure.

6. The surgical tool of claim 1 wherein the bi-stable latch assembly comprises a first segment coupled to a second segment at a j oint, and wherein the movement of the lever causes the first segment to move about the joint relative to the second segment and moves the joint to a position over or under the boundary line.

7. The surgical tool of claim 6 wherein a positon of the joint above the boundary line causes the bi-stable latch assembly to latch the lever in the closed position.

8. The surgical tool of claim 6 wherein a position of the joint below the boundary line causes the bi-stable latch assembly to unlatch the lever.

9. The surgical tool of claim 1 wherein the lever is latched in the closed position only by the bi-stable latch assembly.

10. The surgical tool of claim 1 wherein the surgical tool is an energy tool and the surgical procedure comprises an energy operation.

11. An energy tool for a surgical robotic system, the energy tool comprising:
a tool grasper operable to perform an energy operation; and
a handle coupled to the tool grasper and comprising a lever operable to actuate the tool grasper to perform the energy operation in a first mode in which the lever is latched in a closed position and a second mode in which the lever is unlatched.

12. The energy tool of claim 11 wherein the lever pivots about a first pivot point and is coupled to a latch assembly that pivots about a second pivot point, and wherein a position of the latch assembly relative to a boundary line intersecting the first pivot point and the second pivot point latches the lever in the first mode or unlatches the lever in the second mode.

13. The energy tool of claim 12 wherein positioning of the latch assembly over the boundary line latches the lever in the closed position and actuates the tool grasper to perform the energy operation.

14. The energy tool of claim 12 wherein positioning of the latch assembly under the boundary line unlatches the lever allowing the lever to transition between the closed position and an open position.

15. The energy tool of claim 12 further comprising a lever adjustor coupled to the handle that prevents the latch assembly from latching in the second mode.

16. The energy tool of claim 15 wherein the lever adjustor is a bar that is operable to translate between a first position that allows the latch assembly to latch the lever in the first mode and a second position that prevents the latch assembly from latching the lever in the second mode.

17. The energy tool of claim 15 further comprising an energy application switch coupled to the handle that, when actuated, causes the tool grasper to perform the energy operation.

18. The energy tool of claim 17 wherein the lever adjustor bar in the second position is aligned with the energy application switch and a movement of the lever about the pivot point to the closed position causes the lever adjustor bar to contact the energy application switch and actuate the tool grasper to perform the energy operation.

19. The energy tool of claim 17 further comprising a third mode in which the energy application switch is directly controlled by the user to cause the tool grasper to perform the energy operation when the lever is unlatched.

20. The energy tool of claim 11 further comprising a selection lever coupled to the housing to allow a user to select between the first mode and the second mode.
